(19) United States
(12) Patent Application Publication
(10) Pub. No.: US 2023/0301519 A1
(43) Pub. Date: Sep. 28, 2023

(54) MULTI-MODALITY MEDICAL SYSTEM AND ASSOCIATED DEVICES AND METHODS

(71) Applicant: KONINKLIJKE PHILIPS N.V., EINDHOVEN (NL)

(72) Inventor: Yang SUN, RANCHO CORDOVA, CA (US)

(21) Appl. No.: 18/015,932

(22) PCT Filed: Jul. 20, 2021
(86) PCT No.: PCT/EP2021/070194

§ 371 Date: Jan. 12, 2023

Related U.S. Application Data
(60) Provisional application No. 63/056,142, filed on Jul. 24, 2020.

(51) Int. Cl. A61B 5/00; A61B 8/12

(52) U.S. Cl. CPC A61B 5/0033; A61B 8/12; A61B 8/44

(57) ABSTRACT

A multi-modality medical system an intravascular catheter or guidewire configured to output intravascular data, an external ultrasound probe configured to output digital ultrasound data, and a processor disposed within a multi-modality housing and configured for communication with the external ultrasound probe and the intravascular catheter or guidewire. The external ultrasound probe includes ultrasound-specific hardware, such as signal processing circuitry, sufficient to generate digital ultrasound data that can be processed into images. In an external ultrasound data acquisition mode, the processor is configured to control the external ultrasound probe to generate the digital ultrasound data, and output an external ultrasound image to a display. In an intravascular data acquisition mode, the processor is further configured to control the intravascular catheter or guidewire to generate the intravascular data, and output a graphical representation of the intravascular data to the display.

### TECHNICAL FIELD

The present disclosure relates generally to the assessment and treatment of anatomical features using external ultrasound and additional medical modalities. For example, some embodiments of the present disclosure are suited for obtaining imaging and diagnostic information of different medical modalities to assess the severity of a blockage or other restriction to the flow of fluid through a vessel, such as a stenosis of a human blood vessel.

### BACKGROUND

Innovations in diagnosing and verifying the level of success of treatment of disease have progressed from solely external imaging processes to include internal diagnostic processes. In addition to traditional external image techniques such as X-ray, MRI, CT scans, fluoroscopy, and angiography, small sensors may now be placed directly in the body. For example, diagnostic equipment and processes have been developed for diagnosing vasculature blockages and other vasculature disease by means of ultra-miniature sensors placed on the distal end of a flexible elongate member such as a catheter, or a guide wire used for catheterization procedures. For example, known medical sensing techniques include intravascular ultrasound (IVUS), forward looking IVUS (FL-IVUS), fractional flow reserve (FFR) determination, instantaneous wave-free ratio (iFR) determination, coronary flow reserve (CFR) determination, optical coherence tomography (OCT), trans-esophageal echocardiography (TEE), transthoracic echocardiography (TTE), and image-guided therapy. Traditionally, many of these procedures are carried out by a multitude of physicians and clinicians, where each performs an assigned task. Further, many intraluminal medical modalities require or benefit from other modalities, such as external imaging. For example, an intravascular diagnostic procedure may be guided using an external imaging modality.

One exemplary type of procedure involves pressure measurements within a blood vessel. Currently accepted techniques for assessing the severity of a stenosis in the blood vessel, such as ischemia causing lesions, includes fractional flow reserve (FFR) and instantaneous wave-free ratio (iFR). FFR and iFR are calculations of a ratio of a distal pressure measurement (taken on the distal side of the stenosis) relative to a proximal pressure measurement (taken on the proximal side of the stenosis). FFR and/or iFR provide an index of stenosis severity that allows determination as to whether the blockage limits blood flow within the vessel to an extent that treatment is required. For example, the normal value of FFR in a healthy vessel is 1.00, while values less than about 0.80 are generally deemed significant and require treatment. Common treatment options include angioplasty and stenting. IVUS imaging or OCT imaging may be used instead of or in addition to FFR determination to obtain radial cross-sectional images of a region of interest in a vessel. For example, a physician can examine the cross-sectional IVUS images to determine the severity of a blockage or lesion, and determine which type of treatment to perform based on the examination of the IVUS images.

To carry out an IVUS imaging procedure or an FFR/iFR procedure, various imaging and medical data modalities may be involved to perform the diagnostic procedure. In this regard, FFR measurements, iFR measurements, and IVUS imaging procedures both involve inserting an elongate instrument such as a catheter or guidewire through an access point of the skin of the patient, and navigating the instrument to a particular location in the vasculature to obtain pressure measurements or imaging data. For example, an external ultrasound probe may be used to guide the insertion of an access needle and/or an intravascular instrument through the skin at the access point, and into the vasculature. X-ray imaging such as angiography and fluoroscopy may then be used to guide the instrument to a specific branch of the coronary arteries that is suspected to have the blockage. Each of these modalities may require specific equipment and hardware that can add clutter to the catheter lab that houses the equipment. Although catheter labs may already include the hardware and equipment for some of the modalities, such as X-ray imaging and intravascular sensing instruments (e.g., pressure-sensing guidewires), adding external ultrasound would involve additional ultrasound processing hardware, such as a mobile console on a rolling cart. Adding the cart to the catheter lab may be difficult, as there may be insufficient space available for such hardware. Additionally, each modality may require its own software that can add complexity to the diagnostic procedures.

### SUMMARY

Embodiments of the present disclosure include systems, devices, and methods that integrate multiple different medical modalities to perform diagnostic and/or therapeutic procedures. In one embodiment, a multi-modality medical system includes a communication interface that is configured to couple to an intravascular device, such as an IVUS imaging catheter or a pressure-sensing guidewire, and an external ultrasound probe. The external ultrasound probe includes the ultrasound-specific hardware, such as signal processing circuitry, sufficient to generate digital ultrasound data that can be processed into images. In one aspect, the digital ultrasound data is in a format and condition to be converted into images using non-ultrasound-specific hardware, such as a central processing unit of a computer. A processor or controller controls the external ultrasound probe via the communication interface to generate the digital ultrasound data, and the intravascular device to generate the intravascular data. Because the ultrasound-specific hardware is contained in the ultrasound probe itself, the processor is configured to control both modalities without adding additional external ultrasound-specific hardware (e.g., an ultrasound console or rolling cart) into the catheter lab. Additionally, the processor can be configured with software to conveniently switch between the modalities using a common graphical interface.

According to one embodiment of the present disclosure, a multi-modality medical system comprises: an intravascular catheter or guidewire configured to output intravascular data; an external ultrasound probe configured to output digital ultrasound data; and a processor disposed within a multi-modality housing and configured for communication with the external ultrasound probe and the intravascular catheter or guidewire. The processor is configured to, in response to receiving a user input selecting an external ultrasound data acquisition mode, control the external ultrasound probe to generate the digital ultrasound data, and output, to a display in communication with the processor, an external ultrasound image based on the digital ultrasound data. The processor is further configured to, in response to receiving a user input selecting an intravascular data acquisition mode, control the intravascular catheter or guidewire to generate the intravascular data, and output, to the display, a graphical representation based on the intravascular data.

In some embodiments, the system further includes a communication interface communicatively coupled to the processor, wherein the communication interface comprises a housing and at least one data bus connector, wherein the communication interface is configured to be coupled to the external ultrasound probe and an intravascular catheter or guidewire configured to obtain intravascular data. In some embodiments, the system further includes the display. In some embodiments, the communication interface is configured to be spaced from the processor. In some embodiments, the external ultrasound probe is coupled to the communication interface by an industry standardized data bus. In some embodiments, the industry standardized data bus is a universal serial bus (USB) connection.

In some embodiments, the external ultrasound probe comprises an ultrasound transducer array and signal processing circuitry disposed within a housing. In some embodiments, the signal processing circuit is configured to receive analog signals from the ultrasound transducer array, and generate the digital ultrasound data based on the received analog signals. In some embodiments, the digital ultrasound data is configured to be processed into images and displayed using non-ultrasound-specific hardware.

In some embodiments, the signal processing circuitry of the external ultrasound probe comprises an analog-to-digital converter and a beamformer. In some embodiments, the signal processing circuitry is configured to process raw ultrasound signals to generate the digital ultrasound data such that the digital ultrasound data is in an industry standard image format or video format. In some embodiments, the signal processing circuitry is configured to perform at least one of envelope detection, log compression, or scan conversion. In some embodiments, the external ultrasound probe further comprises a data bus connector coupled to the external ultrasound probe by a cable, wherein the cable is permanently connected to the housing of the external ultrasound probe. In some embodiments, the external ultrasound probe further comprises: a data bus connector coupled to the housing of the external ultrasound probe; and a cable comprising a matching data bus connector configured to removably couple to the data bus connector coupled to the housing.

In some embodiments the system further comprises a user interface device, wherein the user interface device comprises a further display. In some embodiments, the processor is configured to cause the further display to display a graphical user interface that includes a first toggle associated with the external ultrasound data acquisition mode, and a second toggle associated with the intravascular data acquisition mode. In some embodiments, the user interface device is configured to generate a user input signal based on a selection by a user of the first toggle or the second toggle. In some embodiments, the system further includes an X-ray imaging device. In some embodiments, the processor is configured to: receive a further user input selecting an X-ray imaging mode; and in response to receiving the further user input: control the X-ray imaging device to generate X-ray image data; and output, to the display, an X-ray image based on the X-ray image data.

In some embodiments, the processor is further configured to co-register the intravascular data with the X-ray image data. In some embodiments, the further display comprises a touch screen display configured to receive the selection from the user. In some embodiments, the processor is configured to cause the display and the further display to simultaneously display the graphical representation based on the intravascular data. In some embodiments, the intravascular catheter or guidewire comprises an intravascular ultrasound (IVUS) imaging catheter, and wherein the intravascular data comprises IVUS images. In some embodiments, the intravascular catheter or guidewire comprises at least one of an intravascular pressure sensor or an intravascular flow sensor, and wherein the intravascular data comprises at least one of pressure data or flow data.

According to another embodiment of the present disclosure, a multi-modality medical system comprises: a hand-held external ultrasound probe comprising: a first housing configured to be grasped by a human hand; an ultrasound transducer array coupled to the first housing; and signal processing circuitry disposed within the first housing, wherein the signal processing circuit is configured to receive analog signals from the ultrasound transducer array, and generate digital ultrasound data based on the received analog signals, wherein the digital ultrasound data is configured to be processed into images and displayed using non-ultrasound-specific hardware. The system further comprises: a first standard bus connector; a touch screen interface device; a display; a communication hub comprising: a second housing; a second standard bus connector coupled to the second housing, wherein the second standard bus connector is configured to be coupled to the first standard bus connector of the external ultrasound probe; and an intravascular modality bus connector coupled to the second housing, wherein the intravascular modality bus connector is configured to be coupled to an intravascular device. The system further comprises a processing system comprising a processor and a memory, wherein the processing system is configured to be coupled to the communication hub, the touch screen interface device, and the display, wherein the processing system is in communication with the external ultrasound probe and the intravascular device via the communication hub. The memory comprises instructions executable by the processor to: control the external ultrasound probe to generate the digital ultrasound data; cause at least one of the touch screen interface device or the display to display ultrasound images based on the digital ultrasound data; receive a user input signal from the touch screen interface device, wherein the user input signal represents a selection of an intravascular data acquisition mode; and in response to receiving the user input signal: control the intravascular device to generate intravascular data; and cause at least one of the touch screen interface device or the display to display a graphical representation of the intravascular data.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a perspective diagrammatic view of a self-sufficient ultrasound probe that includes signal processing circuitry, according to embodiments of the present disclosure.

FIG. 2 is a diagrammatic view of a multi-modality medical system, according to embodiments of the present disclosure.

FIG. 3 is a diagrammatic schematic view of a multi-modality medical system, according to embodiments of the present disclosure.

FIG. 4 is a perspective view of a bedside communication interface device, according to embodiments of the present disclosure.

FIG. 5 is a perspective view of a bedside controller releasably mounted on an bed rail, according to embodiments of the present disclosure.

FIG. 6 is a diagrammatic schematic view of the bedside controller of FIG. 5, according to embodiments of the present disclosure.

FIG. 7 is a diagrammatic schematic view of a multi-modality medical system that includes a touch-screen control interface, according to embodiments of the present disclosure.

FIG. 8 is a perspective view of a mobile multi-modality diagnostic cart, according to embodiments of the present disclosure.

FIG. 9 is a diagrammatic schematic view of a mobile multi-modality diagnostic cart, according to embodiments of the present disclosure.

FIG. 10 is a perspective view of a clamshell-style mobile multi-modality medical system, according to embodiments of the present disclosure.

FIG. 11 is a screen display of a graphical user interface of a multi-modality medical system, according to embodiments of the present disclosure.

FIG. 12 is a diagrammatic schematic view of a processor circuit, according to embodiments of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As described above, diagnostic procedures and/or therapeutic procedures may involve several different imaging and physiological measurement modalities. For example, to obtain functional flow-related measurements (e.g., fractional flow reserve (FFR), instantaneous wave-free ratio (iFR)), a pressure-sensing catheter and/or guidewire is inserted into the vasculature of the patient. In order to guide the catheter or guidewire to the location of interest, external ultrasound is used to guide the insertion of a needle through an access site on the patient’s skin, and into a blood vessel. Once the catheter or guidewire has been inserted through the needle and into the vasculature, the physician may switch to an X-ray imaging modality, such as fluoroscopy, to guide the catheter or guidewire to a location associated with a potential blockage or lesion. For example, fluoroscopy may be used to guide the catheter or guidewire to a particular branch of the coronary arteries in which a stenosis has been identified. Next, the physician may initiate the pressure-sensing catheter or guidewire to perform a pull-back procedure to obtain a plurality of pressure measurements both distal and proximal to the stenosis to obtain the functional flow-related measurements. Additionally or alternatively, an IVUS imaging catheter may be guided to the region of interest in the vasculature to perform an imaging pull-back procedure to obtain a plurality of intravascular cross-sectional images of the stenosis.

Each of these medical modalities requires specific hardware and processing equipment that take up space in the operating environment or catheter lab. Adding external ultrasound to a catheter lab, which may already include hardware for X-ray imaging, intraluminal pressure or flow measurements, and IVUS imaging, may be inconvenient or impractical. In this regard, conventional external ultrasound systems often include a console or mobile cart that carries ultrasound-specific hardware to control the ultrasound probe and process ultrasound signals or data into image data or images that can be displayed. There may be limited available space in the catheter lab for the console or cart. Additionally, external ultrasound modalities typically include their own software applications and interfaces to control the imaging procedure and display the images. Accordingly, switching between external ultrasound and other modalities may be inconvenient and complicated.

The present disclosure provides systems, methods, and associated devices that allow external ultrasound to be integrated into a multi-modality medical system in a spatially efficient and convenient way. Embodiments of the present disclosure may improve diagnostic and imaging workflows, as little or no processing equipment for the external ultrasound is added, and the same equipment (e.g., display, communication interface) used for the intravascular modalities can be used for external ultrasound. In this regard, an ultrasound probe that includes the ultrasound-specific hardware for producing digital image data and/or images that can be output to a display using non-ultrasound-specific hardware of the multi-modality system. Further, the present disclosure describes software and graphical user interfaces that improve diagnostic workflows by allowing for easy transitions between the modalities using an interface device.

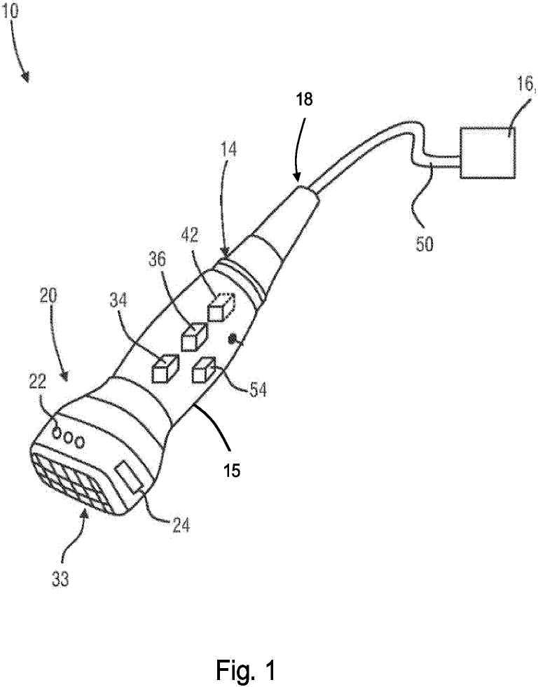

FIG. 1 shows an embodiment of a self-sufficient ultrasound imaging apparatus 10 having an ultrasound probe 14 that can be used in a medical diagnostic environment (e.g., a catheter lab) that adds little or no additional hardware to a multi-modality medical system. The probe 14 has a probe housing 15 that includes all ultrasound-specific imaging hardware, including a transducer array 33 coupled to a probe head portion 20, an analog-to-digital converter (ADC) 34, a beamformer 54, a signal processor 36 and, optionally, an image processor 42. In some embodiments, the housing 15 of the ultrasound probe 14 is configured to grasped by a user’s hand. In some embodiments, the ultrasound probe 14 is configured to be worn by the patient using a strap or adhesive. Further, the probe housing 15 may include a first input device having, for example, a button 24 to control the image acquisition. Further, an output device 22 may be provided on the probe, e.g. in the form of a light emitting diode (LED) or a plurality of lights or LEDs 22. The probe 14 is connected via an interface 50 to a mobile console. In some embodiments, the probe 14 may be configured to process raw ultrasound signals obtained by the elements of the array 33 to provide two-dimensional and/or three-dimensional ultrasound images in a format usable by conventional computing devices without using additional ultrasound-specific signal processing or control hardware.

The signal conditioning and processing circuitry within the probe 14 is configured to perform a variety of functions for the ultrasound signal, including band pass filtering, analog-to-digital converting, beamforming, noise filtering, low-pass filtering, spatial filtering, log compression, envelope detection, scan conversion, and/or other signal processing functions. For example, raw analog signals may be filtered by a filter of the signal processing circuitry. The analog ultrasound signals may include backscatter signals generated by the intraluminal ultrasound device at a variety angular positions (e.g., at 0, 2×θ, 3×θ, ...) around a circumference of the device. The filtering performed may include, for example, band pass filtering, noise filtering, etc. The ADC 34 receives the filtered analog ultrasound signal from the filter and samples the analog ultrasound signal to provide digital ultrasound signals or data. The digital ultrasound signals or data may include a plurality of scanlines each including a sequence of real-valued radiofrequency (RF) samples along an imaging depth (e.g., a radial axis of the imaging device) as described in greater detail herein.

The digital ultrasound signals are then passed to the beamformer (BF) 54 configured to perform a coherent delay-and-sum operation on the image signals to provide beamformed signals. In some aspects, the BF 54 may perform a baseband conversion and/or demodulation on the image signals. In some embodiments, the BF 54 may include a rectifier configured to convert the real-valued RF samples in the image signals to baseband (BB) signal signals including complex in-phase, quadrature-phase (IQ) pairs. The rectifier may perform down-conversion, low-pass filtering, and/or decimation. The down-conversion converts the RF output signal data from the RF to BB, for example, by down-mixing the RF signals with two sinusoidal signals with a 90 degrees phase difference.

The beamformed signals are then passed to a signal processor 36, which may include a memory device configured to temporarily store the beamformed image signals for further processing. The signal processor 36 may comprise a volatile memory resource that is either accessible to a single processing unit (e.g., CPU core or FPGA) or a shared memory accessible to multiple processors (e.g., multiple cores, GPU, and/or multiple paths within an FPGA). In some embodiments, the signal processor 36 comprises a duplicator configured to duplicate the image signals to be processed along different processing paths. As described further below, the processing paths may be configured to perform operations on the image signals simultaneously or at different times. The signal processor 36 may include filtering circuitry to perform additional filtering on the signals. In that regard, the signal processor 36 may be configured to perform different filtering operations (e.g., band pass filtering, low pass filtering) on the signals to provide different filtered signals.

The digital beamformed signals may then be passed to a log compression module of the signal processor 36 that performs log compression. In particular, the log compression module may be configured to reduce the dynamic range of the image signals for efficient display. For example, the dynamic range of the image signals may be mapped to a logarithmic curve. In some examples, the log compression module may perform the mapping based on a table lookup, where the table may be encoded with a log compression curve. In some embodiments, the signal processor 36 performs an envelope detection operation before or after the log compression.

The signal processor 36 may further include a scan conversion module coupled to the log compression module and configured to perform scan conversion on the image signals output by the log compression module to produce image data in a suitable display format. In an example, the image data may be in polar coordinates and the scan conversion module may convert the image data into Cartesian coordinates for display. An optional image processor 42 is also included in the ultrasound probe 14, and includes circuitry for processing the digital ultrasound data into image data that can be displayed on a display device. In this regard, the image processor may be configured to process the ultrasound data into a format usable by conventional display devices. For example, the image processor may be configured to provide images in an industry standard image format or video format, such as .JPG, .MOV, .MP4, .PNG, .TIF, .RAW, .AVI, .WMV, .FLV, or any other suitable format.

Accordingly, the probe 14 provides a self-sufficient ultrasound imaging device that can be used with conventional computing and display devices without additional ultrasound-specific hardware. Further, the interface 50, which includes a cable and connector 16, may be an industry standard connector, such as Universal Serial Bus (USB), Ethernet, RS-232, Firewire, Serial Peripheral Interface (SPI), Peripheral Component Interconnect (PCI), Serial AT Attachment (Serial ATA), Serial small computer system interface (SCSI), Video Graphics Array (VGA), Digital Visual Interface (DVI), High-Definition Multimedia Interface (HDMI), or any other suitable interface standard. In this regard, the probe 14 may be configured to be used with a conventional, off-the-shelf computing device, such as a personal computer, mobile laptop computer, smartphone, tablet, or any other suitable computing device. In some embodiments, the interface 50 may comprise a cable that is permanently attached to the probe 14. In some embodiments, the interface 50 comprises a cable that is removably coupled to the probe 14. For example, the probe 14 may comprise a data bus connector 18 at a proximal end of the housing 15 of the probe 14 that can releasably couple to a corresponding connector of the interface 50. In other embodiments, the probe 14 comprises a wireless communication interface configured to communicate wirelessly with a processing system and/or interface device. For example, the probe 14 may comprise a wireless transceiver configured to receive and transmit information according to a wireless communications protocol, including Bluetooth®, Wi-Fi, UWB, ZigBee, Cellular, or any other suitable wireless protocol. As explained below, a multi-modality medical system may advantageously integrate external ultrasound as explained above without including additional ultrasound-specific hardware, display devices, and/or interface devices, which can complicate and add clutter to medical diagnostic environments.

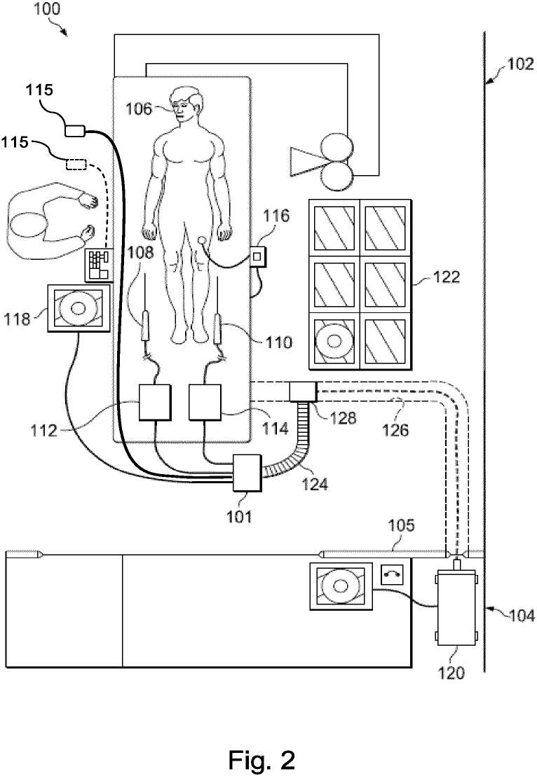

FIG. 2 is a schematic drawing depicting a multi-modality medical system 100 including a powered medical communication hub 101. The system 100 is a data collection solution for multiple modality medical sensing. Generally, in the system 100, the hub 101 is a central unit that connects to a plurality of medical sensing-related tools, distributes power to the plurality of tools, and facilitates communication between the tools and a processing workstation and/or data network. In one embodiment, the communication system 100 may be utilized to collect data from medical sensing devices and transmit it to computing resources, where it is processed and returned.

In the illustrated embodiment, the medical sensing communication system 100 is deployed in a catheter lab 102 having a separate control room 104 isolated by an intervening wall 105. In other embodiments, however, the medical sensing communication system 100 may be deployed in an operating room, diagnostic room, or other medical environment used to perform any number of patient procedures. The catheter lab 102 includes a sterile field but its associated control room 104 may or may not be sterile depending on the requirements of a procedure and/or health care facility. The catheter lab and control room may be used to perform on a patient any number of medical sensing procedures such as angiography, fluoroscopy, external ultrasound, intravascular ultrasound (IVUS), forward looking IVUS (FL-IVUS), fractional flow reserve (FFR) determination, instantaneous wave-free ratio (iFR), a coronary flow reserve (CFR) determination, optical coherence tomography (OCT), trans-esophageal echocardiography (TEE), transthoracic echocardiography (TTE), virtual histology (VH), intravascular photoacoustic (IVPA) imaging, a functional measurement determination, computed tomography, intracardiac echocardiography (ICE), forward-looking ICE (FLICE), intravascular palpography, or any other medical sensing modalities known in the art. For example, in catheter lab 102 a patient 106 may be undergoing a multi-modality procedure, in which IVUS data will be collected with an IVUS catheter 108 and OCT data will be collected with an OCT catheter 110. The IVUS catheter 108 may include one or more sensors such as a phased-array transducer. In some embodiments, the IVUS catheter 108 may be capable of multi-modality sensing such as IVUS and IVPA sensing. The OCT catheter 110 may include one or more optical sensors.

Additionally, an external ultrasound probe 115 is coupled to the hub 101. The external ultrasound probe 115 may include a beamformer and signal processing components such that the system 100 can provide images on the display devices 122, 118, without additional ultrasound-specific hardware. Alternatively or additionally, the ultrasound probe 115 may be coupled to an interface of the bedside control surface 118. The ultrasound probe 115 may connect to the hub 101 and/or the bedside control surface 118 using any suitable form of wired and/or wireless communication as described herein. For example, in an exemplary embodiment, the ultrasound probe 115 may connect to the hub 101 and/or the bedside control surface 118 by an industry standard wired interface, such as USB, VGA, HDMI, or any other suitable communication standard. In other embodiments, the ultrasound probe 115 may connect to the hub 101 and/or the bedside control surface 118 using an industry standard wireless interface, such as Bluetooth®, ultra-wide band (UWB), Wi-Fi, ZigBee, or any other suitable interface.

The system 100 further includes a number of interconnected medical sensing-related tools in the catheter lab 102 and control room 104 to facilitate this multi-modality workflow procedure, including an IVUS patient interface module (PIM) 112, an OCT PIM 114, an electrocardiogram (ECG) device 116, a bedside control surface 118, a processing system 120, and a boom display 122. The hub 101 in the catheter lab 102 consolidates the multitude of cables extending from these medical sensing-related tools and communicatively couples them to the processing system 120. That is, the hub 101 is an intermediary through which the tools in the catheter lab 102 connect to the processing system 120. In general, the hub 101 is coupled to the processing system 120 via a plurality of power and communication cables. To alleviate the problems associated with loose cabling in a crowded medical working environment, the cables coupling the hub 101 to the processing system 120 extend through a protective hose 124 and a trench 126 in the floor of the catheter lab 102. The cables enter the trench 126 through a trench entry port 128. In this manner, the cables are aggregated and protected the entirety of the distance from the hub 101 to the processing system 120. Of course, the cabling between the hub 101 and processing system 120 may be oriented in many other configurations depending on the specific catheter lab configuration. For instance, the cabling may extend through the protective hose 124 and enter a wall or a ceiling through a termination plate before travelling to the processing system. In the illustrated embodiment, the hub 101 is mounted on the floor near the patient 106 to reduce the amount of cabling located in high-traffic areas near the patient. In some instances, the hub 101 may be located in the sterile field surrounding the patient 106.

In the illustrated embodiment, the processing system 120 is a computer workstation with the hardware and software to acquire, process, and display multi-modality medical sensing data, but in other embodiments, the processing system 120 may be any other type of computing system operable to process medical data or assist in computer aided surgery (CAS). In the embodiments in which processing system 120 is a computer workstation, the system includes at least a processor such as a microcontroller or a dedicated central processing unit (CPU), a non-transitory computer-readable storage medium such as a hard drive, random access memory (RAM), and/or compact disk read only memory (CD-ROM), a video controller such as a graphics processing unit (GPU), and a network communication device such as an Ethernet controller. In some embodiments, the processing system 120, comprises a single processing component or chip and/or is disposed within a single housing. In some embodiments, the processing system comprises multiple processing components or chips. In some embodiments, the processing system is disposed within multiple housings, which may be separate and placed in different locations. U.S. Pat. Application No. 61/473,570, entitled “MULTI-MODALITY MEDICAL SENSING SYSTEM AND METHOD”, discloses a computing resource capable of processing multi-modality medical sensing data and is hereby incorporated by reference in its entirety.

As mentioned above, the ECG device 116 is also communicatively coupled to hub 101 via a wired or wireless connection. The ECG device 116 is operable to transmit electrocardiogram signals from patient 106 to the hub 101. In some embodiments, the hub 101 may be operable to synchronize data collection with the catheters 108 and 110 using the ECG signals from the ECG 116.

The bedside control surface 118 is also communicatively coupled to the hub 101 and provides user control and display of the particular medical sensing modality (or modalities) being used to diagnose the patient 106. In the current embodiment, the bedside control surface 118 is a touch screen that provides user controls and diagnostic images on a single surface. In alternative embodiments, however, the bedside control surface 118 may include both a non-interactive display and separate controls such as physical buttons and/or a joystick. In the illustrated embodiment, the bedside control surface 118 and hub 101 communicate over a wired connection such as a standard copper link but, alternatively, the control surface 118 and hub 101 may communicate wirelessly. The bedside control surface 118 includes an integrated processing unit to drive a graphical user interface (GUI)-based workflow presented on the touch screen. U.S. Pat. Application No. 61/473,591, entitled “DISTRIBUTED MEDICAL SENSING SYSTEM AND METHOD” and filed on Apr. 8, 2011 under attorney docket number 44755.784, discloses a bedside control surface that executes GUI-based workflows using a software framework and is hereby incorporated by reference in its entirety.

The system 100 further includes a boom display 122. The boom display 122 may include one or more monitors capable of displaying information associated with a medical sensing procedure. In the illustrated embodiment, the boom display 122 is coupled to, powered, and driven by the hub 101. The boom display 122 is configured to display images and/or data obtained by one or more of the modalities coupled to the system. For example, the processing system 120 may drive the boom display 122 to display external ultrasound images obtained by the ultrasound probe 115 to guide the insertion of a needle into the vasculature of the patient 106, and to guide placement of an intravascular device (e.g., 110, 108) into the vasculature. Further, the processing system 120 and/or the bedside control surface 118 may be used to control the boom display 122 to show intravascular images and/or physiological data obtained by the intravascular devices.

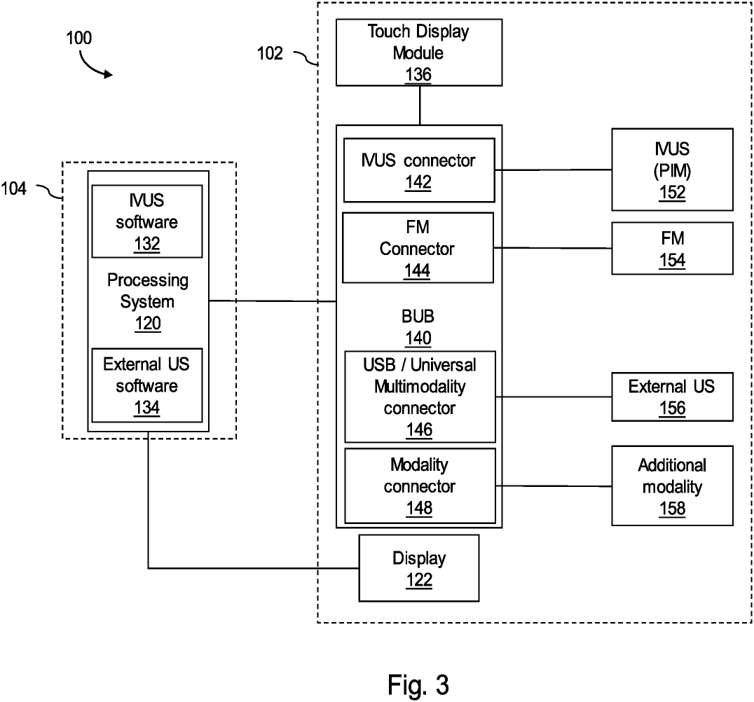

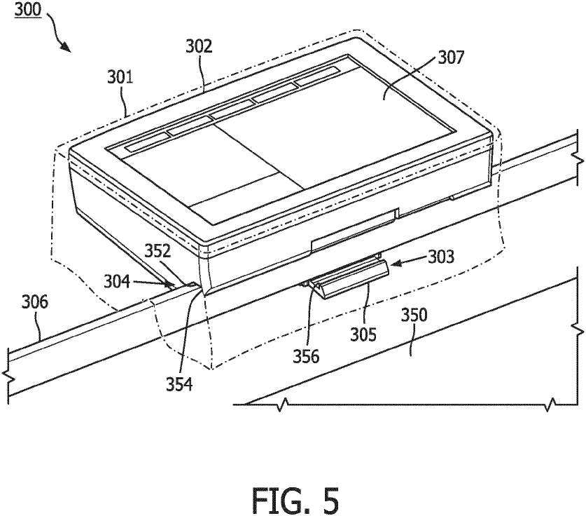

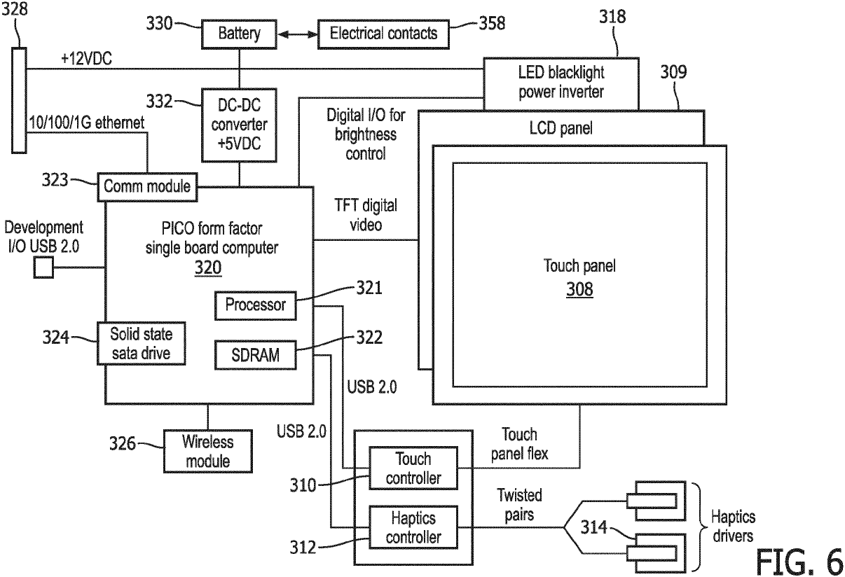

FIG. 3 is a diagrammatic schematic view of a multi-modality medical system, according to embodiments of the present disclosure. The system 100 is distributed between a sterile medical diagnostic/therapeutic environment, such as a catheter lab 102, and a control room 104. A variety of components are positioned within the catheter lab 102, including a communication hub or bedside unit box (BUB) 140, a touch display module 136, a boom display 122, an IVUS device, such as an IVUS PIM 152, a functional measurement (FM) device 154, an external ultrasound probe 156, and an additional modality 158. The touch display module 136 is coupled to the processing system 120 via the BUB 140. In other embodiments, the touch display module 136 is in direct communication with the processing system 120, without being coupled through the BUB 140. The touch display module 136 may include a touch screen, as explained further below with respect to FIGS. 5 and 6. The touch screen may be used to control the various modalities available in the catheter lab 102, including imaging procedures, such as angiography, fluoroscopy, external ultrasound, IVUS, and OCT, and physiological measurements, such as pressure, flow, FFR, iFR, or any other suitable physiological measurement. The touch display module 136 may be configured to be spaced from the processing system 120. For example, the touch display module 136 may be portable, and movable throughout the catheter lab 102.

The BUB 140, which may also be referred to as a communication interface or a communication hub, is in communication with the processing system 120, which includes software for controlling the IVUS imaging procedure (e.g., initiate imaging protocol, adjust imaging parameters), and external ultrasound software to control the external ultrasound imaging procedure. However, it will be understood that the processing system 120 may include software for controlling any of the modalities described herein. In some aspects, the IVUS software 132 and the external ultrasound software 134 may comprise modules or parts of a single executable application. In other embodiments, the IVUS software 132 and the external ultrasound software 134 comprise different applications that can be activated independently of one another. The processing system 120 may be configured to receive data associated with the various medical modalities, and prepare screen displays and/or graphical user interfaces to be displayed on the boom display 122, for example. Additionally, the touch display module 136 may be configured to display images or data that is generated, processed, and/or manipulated by the processing system 120.

The BUB 140 includes a plurality of connectors for the plurality of medical imaging and diagnostic modalities. In this regard, the BUB 140 includes an IVUS connector 142, an FM connector 144, a universal multi-modality connector 146, and an additional modality connector 148, each connected to their corresponding devices. The BUB 140, which is further described below, provides a convenient location for connecting diagnostic equipment and instruments, such as the IVUS device 152, the functional measurement device 154, the external ultrasound device 156, and the additional modality 158. In this regard, the BUB 140 may be attached to the bed in the catheter lab 102 or positioned on a surface near the bed. The additional modality device 158 may correspond to any suitable medical modality, including external ultrasound, FL-IVUS, FFR determination, iFR determination, CFR determination, OCT, TEE, TTE, IVUS, VH, IVPA imaging, computed tomography, ICE, FLICE, a therapeutic device, or any other suitable medical modality.

The IVUS connector 142, FM connector 144, and additional modality connector 148 may comprise specialized, proprietary connectors for their corresponding instruments. By contrast, the USB or universal multi-modality connector 146 is an industry standard connector that may connect to a wide variety of medical devices, input devices, flash memory drives, or any other suitable peripheral device. As explained further below, the USB connector 146 may be used to connect an external ultrasound probe 156 to the system 100. For example, the probe 14 explained above, which may include a USB connector configured to interface with the connector 146, can be coupled to the system 100 without additional consoles, interface devices, or processing equipment.

In operation, the processing system 120 may be in communication with the touch display module 136 and/or the boom display 122 to control the operations of the various modalities, including switching from one or more medical modalities to one or more different medical modalities. For example, the processing system may be configured to control the external ultrasound probe 156 to generate digital ultrasound data. In some aspects, controlling the external ultrasound probe 156 may include executing a software application comprising instructions to perform an external ultrasound imaging protocol, and output, to a display, a graphical user interface (GUI) that includes graphical interface objects (buttons, tabs, toggles, windows) associated with one or more medical modalities. The GUI may also include an observation window that shows the ultrasound images obtained by the ultrasound probe 156. In some embodiments, controlling the ultrasound probe 156 to generate the digital ultrasound data comprises using an interface device, such as a physical button, on the probe, or on a user interface device of the system 100, such as the touch display module 136. The processing system may then cause a display, such as the boom display 122 or the touch display module 136, to display ultrasound images based on the digital ultrasound data. In some embodiments, the ultrasound images comprise two-dimensional images and/or three-dimensional ultrasound images. In some embodiments, the images comprise B-mode ultrasound images, M-mode images, bi-plane images, and/or any other suitable type of image. In some embodiments, the processing system 120 is configured to generate the ultrasound images based on the digital ultrasound data obtained by the ultrasound probe 156. In some embodiments, the digital ultrasound data output by the ultrasound probe 156 is in a format and condition ready for display by a conventional display device.

To switch to a different modality, the processing system 120 may receive a user input signal from a user input device, such as the touch display module 136. The user input represents a selection of an intravascular data acquisition mode. In some embodiments, the input is generated based on a user selection a GUI object (e.g., tab, button, slider, drop-down menu) on a GUI of the software application. In other embodiments, the input comprises an instruction to initiate or launch a different software application associated with the different medical modality. In some embodiments, switching from a first medical modality to a second medical modality comprises deactivating the first medical modality and initializing the second medical modality.

For example, the user input may comprise an instruction to switch from an external ultrasound modality to an intravascular modality, such as IVUS or FM. In response to receiving the user input, the processing system controls the intravascular device to generate intravascular data and causes the display to display a graphical representation of the intravascular data. In some embodiments, the processing system is configured to display the graphical representation of the intravascular data simultaneously with data from a different modality, such as an x-ray imaging modality. In some embodiments, the graphical representation of the intravascular data comprises an image obtained by an intravascular imaging device, such as an IVUS imaging catheter or an OCT imaging catheter. In other embodiments, the graphical representation comprises a plot of physiological measurements co-registered with positional data of the vessel. In some embodiments, the graphical representation comprises a waveform of pressure and/or flow data. In some embodiments, the graphical representation illustrates one or more FM values (e.g., FFR, iFR) calculated for a plurality of locations within the vessel. In some embodiments, the FM values are co-registered with the X-ray images. For example, the FM values may be overlaid on an angiographic image of a vessel at locations corresponding to the FM values.

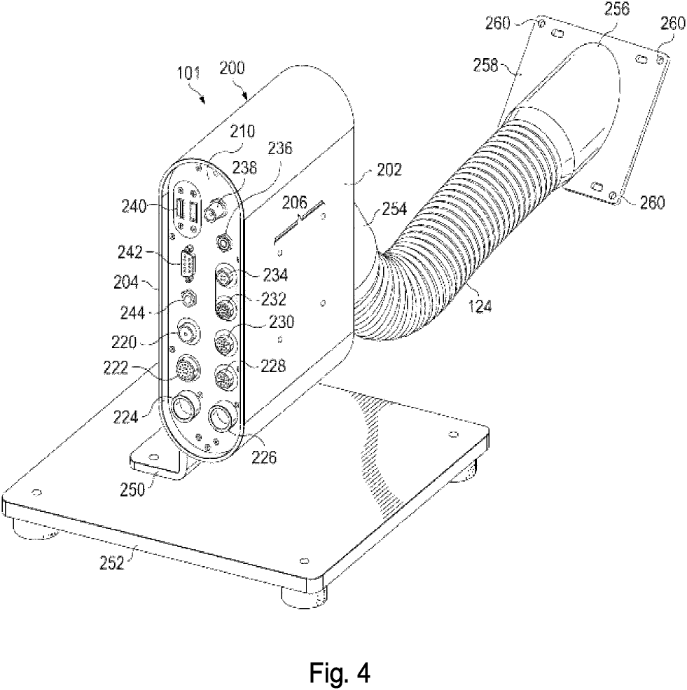

FIG. 4 is a perspective view of an embodiment of a powered medical communication hub. In some aspects, the BUB 140 may comprise the hub 101 shown in FIG. 4. The hub 101 may include the IVUS PIM connector 220, the functional measurement (FM) tool connector 222, the first and second fiber optic connector 224 and 226, the OCT PIM connector 228, the bedside control surface connector 230, the FLIVUS PIM connector 232, the FLIVUS footswitch connector 234, the auxiliary power connector 236, the ECG/aortic device connector 238, the USB connectors 240, and the VGA display connector 242. The IVIS PIM connector 220 may be communicatively coupled to the processing system 120 via a wired or wireless communication link. A cable transmitting power, ground, and data signals extends from the processing system 120 to the link, where the signals are internally forwarded to the IVUS PIM connector 220.

The hub 101 further includes a MultiFiber Push-On (MPO) link comprising first and second fiber optic connectors 224 and 226. In general, the MPO link is configured to aggregate fiber optic signals and route them over a single fiber optic line. The hub 101 further includes an OCT PIM connector 228 and the bedside control surface connector 230. As mentioned above, the connectors 228 and 230 pass Ethernet-based data signals to connected medical-sensing tools. Thus, in the illustrated embodiment, the connectors 228 and 230 may be coupled to the processing system 120 by RJ45 jacks that respectively accept Cat 5e cables. However, in other embodiments, the connectors 228, 230 can be in communication with the processing system 120 using Ethernet-based data over coaxial, fiber optic, or some other type of suitable cable. The hub 101 further includes an ECG device connector 238 configured to couple to an ECG device, and a VGA connector 242 configured to pass video information to the display 122. In other embodiments, the connector 242 may be configured to accept other video-based connectors such as a DVI connector, an HDMI connector, a DisplayPort connector, or an S-Video connector.

Further, the hub 101 includes USB connectors 240 that may be communicatively coupled to the MPO link 1208. As such, the processing system 120 may communicate with USB-based devices such an input device (e.g. joystick, mouse, keyboard, touchpad etc.) even if the hub 101 is located hundreds of meters from the workstation. Additionally, the FLIUVS PIM connector 232 may pass USB-based data to the processing system 120 via the hub 101. Further, the FLIVUS foot switch connector 234 and the ECG device connector 238 are communicatively coupled to the FLIVUS connector 232 so that signals from a FLIVUS footswitch and the ECG device may be used to coordinate data collection by a FLIVUS PIM coupled to the connector 232.

The USB connectors 240 may be used to connect additional medical modalities to the system, such as the ultrasound probe 14 described above. Accordingly, the processing system 120 and/or the touch display module 136, may be configured to receive ultrasound images or image data from the external ultrasound probe 156 by a USB cable. In this way, external ultrasound may be added to the system simply by plugging the probe into one of the USB connectors 240. Accordingly, in some embodiments, external ultrasound is made available within the catheter lab 102 without adding significant clutter or large equipment. The external ultrasound probe, which is in communication with the processing system 120, can be controlled by the external ultrasound software 134 stored on a memory of the processing system 120.

The hub 101 may include power distribution circuitry configured to distribute power to medical sensing-related tools connected to the hub 101. In some embodiments, a combination of hardware and software is used for power distribution, in which software controls power flow through the hardware. In some embodiments, the hub 101 may be operable to determine the amount of power required by medical sensing-related tool connected to a connector on the hub 101 and dynamically supply the correct amount of power. In yet further embodiments, the hub 101 may include a controller to interrogate newly-connected medical sensing tools to determine operational attributes such as voltage requirements. U.S. Pat. Application No. 61/473,625, entitled “MEDICAL SENSING COMMUNICATION SYSTEM AND METHOD”, discloses a medical sensing communication system that includes a controller and power supply unit that are operable to dynamically supply different medical sensing tools with different amounts of power based on their needs and is hereby incorporated by reference in its entirety. In some embodiments, the processing system 120 provides the hub 101 electrical power.

FIG. 5 is a diagrammatic perspective view of a bedside controller 300 mounted to a bed rail 306, and FIG. 6 is a functional block diagram of the bedside controller 300 according to aspects of the present disclosure. The bedside controller 300 may comprise the bedside control surface 118 and/or the touch display module 136 described above. The controller 300 is operable to, among other things, initiate a medical sensing or treatment procedure workflow, display real-time data (e.g., visual representations of pressure data) obtained during the procedure, and accept user touches on the visual representations of pressure data from a clinician. The bedside controller 300 generally improves system control available to a clinician working at a patient table. For instance, giving a clinician both workflow control and analysis capability within the sterile field reduces errors and improves workflow efficiency.

As show in FIG. 5, the bedside controller 300 includes an integrally formed housing 302 that is easy to grasp and move around a catheter lab or other medical setting. In one embodiment, the integrally formed housing 302 may be seamlessly molded from materials such as thermoplastic or thermosetting plastic or moldable metal. In other embodiments, the integrally formed housing 302 may comprise a plurality of housing portions fixedly bonded in a substantially permanent manner to form an integral housing. The housing 302 is resistant to fluids, and, in one embodiment, may have a rating of IPX4 against fluid ingress as defined by the International Electrotechnical Commission (IEC) standard 60529. In other embodiments in which the housing 302 may be used in different environments, the housing 302 may have a different fluid ingress rating. In the illustrated embodiment, the housing 302 is about 10.5 inches in width, about 8.25 inches in height, and has as thickness of about 2.75 inches. In alternative embodiments, the housing may have a different width, height, or thickness that is similarly conducive to portability.

The housing 302 further includes self-contained mounting structure 303 disposed on the housing 302. In the illustrated embodiment, the mounting structure 303 is disposed near an outer edge of the housing 302. The mounting structure 303 allows the bedside controller 300 to be releasably mounted in a variety of places in and out of a catheter lab in a self-contained manner. That is, the bedside controller 300 may be directly secured to another object without the use of a separate external mount. In the illustrated embodiment, the mounting structure 303 includes a mounting channel 304 and a retaining clamp 305 that pivots over the mounting channel to secure a mounting platform there within. The mounting channel 304 is defined by a longer front wall 350, a top wall 352, and a shorter back wall 354, and the retaining clamp includes a slot 356 that extends through the clamp in a manner generally parallel to the mounting channel. The front wall 350 and the back wall 354 are generally perpendicular to a touch-sensitive display 307 in the housing 302, and the top wall 352 is generally parallel to the display 307. In the illustrated embodiment, the retaining clamp 305 is spring-loaded and releasably exerts pressure on objects situated in the mounting channel. In alternative embodiments, the retaining clamp may be configured differently and exert force via mechanisms other than springs.

In operation, the bedside controller 300 may be releasably secured to a mounting platform, for example a bed rail 306, by pivoting the mounting clamp 305 to an open position, positioning the controller such that the rail extends through the length of the channel 304, and releasing the clamp such that it secures the rail within the channel. When the rail 306 is positioned in the mounting channel 304 and the clamp 305 is holding it therein, three surfaces of the rail are respectively engaged by the front wall 350, the top wall 352, and the back wall 354, and a fourth surface of the rail extends through the slot 356 in the clamp 305. In this manner, the mounting structure 303 may maintain the bedside controller 300 in a position generally parallel to a procedure table 350 associated with the bed rail 306. Described differently, the mounting structure 303 is a cantilevered mounting structure in that it secures one end of the controller to an object while the majority of the controller extends away from the object in an unsupported manner. Such a cantilevered position allows for a display of the controller to be both readable and at a comfortable input angle for an operator. Further, the self-contained mounting structure 303 allows the bedside controller 300 to be quickly released from the bed rail 306 and reattached to an IV pole, a cart on which a processing system is deployed, or other location in or out of the sterile field to allow for convenient workflow control and image analysis. In alternative embodiments the mounting structure 303 of the bedside controller may vary from the design illustrated in FIGS. 5 and 6, and may include additional and/or different components to allow for self-contained mounting.

Referring to FIGS. 5 and 6, embedded into the front of the housing 302 is the touch-sensitive display 307 that comprises both a touch panel 308 and a flat panel display 309. The touch panel 308 overlays the flat panel display 308 and accepts user input via human touch, stylus touch, or some other analogous input method. In other words, the touch-sensitive display 307 displays images and accepts user input on the same surface. In the current embodiment, the touch panel 308 is a resistive-type panel, but in alternative embodiments it may be a capacitive-type panel, projective-type panel, or some other suitable type of touch enabled input panel. Further, the touch panel 308 is operable to accept multiple inputs simultaneously (multitouch), for instance, to enable rotation of a three-dimensional rendering of a vessel along multiple axes. Additionally, the touch panel 308 is capable of receiving input when a sterile drape is covering the bedside controller 300 and also when a user is gloved. The touch panel 308 is controlled by a touch controller 310 disposed within the housing 302. Further, when a clinician makes contact with the touch panel 308, the touch panel is operable to provide haptic feedback via a haptics controller 312 and haptics drivers 314. This haptic technology is operable to simulate a plurality of sensations on the touch panel 308 by varying the intensity and frequency of vibrations generated when a user contacts the touch panel. In some embodiments, the housing 302 may include a sheath configured to store a stylus therein. Thus, a clinician may remove the stylus from the sheath in the housing to make measurements on the bedside controller and store it when the measurements have been completed.

Beneath the touch panel 308 is the flat panel display 309 that presents a graphical user interface (GUI) to a user. In the illustrated embodiment, the flat panel display 309 is a LCD display but in alternative embodiments, it may be a different type of display such an LED display or an AMOLED display. In the illustrated embodiment, the flat panel display 309 is illuminated by a LED backlight power inverter 318. As mentioned above, the GUI not only allows a clinician to control a medical sensing workflow, but also view and interact with pressure data obtained from a patient in the sterile field.

The bedside controller 300 includes a single board processing platform 320 within the housing 302 that is operable to render the GUI 316 and process user touch input. In the illustrated embodiment, the processing platform has a pico form factor and includes integrated processing components such as a processor 321, system memory 322, graphics processing unit (GPU), communications module 323, and I/O bus controller. In some embodiments, the processor 321 may be a low power processor such as an Intel Atom® processor or an ARM-based processor, and the communications module 323 may be a 10/100/1Gb Ethernet module. And, the I/O bus controller may be a Universal Serial Bus (USB) controller. The bedside controller 300 further includes a storage module 324 that is a non-transitory computer readable storage medium operable to store an operating system (i.e. software to render and control the GUI), data and/or visual representation manipulation software, medical sensing data and visual representations received from a processing system, and other medical sensing-related software. The processor 321 is configured to execute software and instructions stored on the storage module 324. In the illustrated embodiment, the storage module 324 is a solid state drive (SSD) hard drive communicatively coupled to the processing platform 320 via a SATA connection, but, in alternative embodiments, it may be any other type of non-volatile or temporary storage module. The bedside controller 300 further includes a wireless communications module 326 communicatively coupled to the processing platform 320. In some embodiments, the wireless communications module is a IEEE 802.11 Wi-Fi module, but in other may be a Ultra Wide-Band (UWB) wireless module, a wireless FireWire module, a wireless USB module, a Bluetooth module, or another high-speed wireless networking module.

In the illustrated embodiment, the bedside controller 300 is powered via both a wired 12VDC power-over-Ethernet (PoE) connection 328 and a battery 330 disposed within the housing 302. In one embodiment, the battery 330 may be sealed within the integrally formed housing 302 and may be recharged through electrical contacts disposed on the exterior of the housing and electrically coupled to the battery. The front wall 350 may include one or more electrical contacts 358 through which the battery 330 may be charged when the controller is mounted to objects with compatible charging structure. In other embodiments, the housing 302 may include a battery compartment with a removable cover to permit battery replacement. Such a battery compartment cover may be resistant to fluid ingress (e.g., with an IPX4 rating). The beside controller 300 may be coupled to a processing system in the catheter lab via the PoE connection 328, over which it receives medical sensing images that have been captured from the patient and rendered on the processing system. In operation, when the bedside controller is coupled to the PoE connection 328, it receives power and communications over the same physical wire. When the bedside controller 300 is disconnected from the PoE connection 328, it runs on battery power and receives data wirelessly via the wireless communications module 326. When used wirelessly in a catheter lab, the beside controller may directly communicate with a processing system (i.e. in an ad-hoc wireless mode), or, alternatively, it may communicate with a wireless network that serves a plurality of wireless devices. In alternative embodiments, the bedside controller 300 may receive power and data through different wired connections, or receive data communications through a wired data connection and power from the battery 330, or receive data communications through the wireless module 326 and power from a wired electrical connection. In some embodiments, the bedside controller 300 may be used in a semi-wireless configuration, in which the battery 330 provides backup power to the controller when the controller is temporarily disconnected from a wired power source. For example, if at the beginning of a procedure, the bedside controller 300 is connected to a PoE connection (or other type of wired connection) and during the procedure the controller must be disconnected from the PoE connection to allow for a cabling adjustment, the battery 330 may keep the controller alive until a PoE connection can be re-established. In this manner, a full power-off and reboot of the controller 300 is avoided during a procedure. As shown in FIG. 6, a DC-DC power converter 332 converts input voltage to a voltage usable by the processing platform 320.

It is understood that although the bedside controller 300 in the illustrated embodiments of FIGS. 5 and 6 includes specific components described herein, the bedside controller 300 may include any number of additional components, for example a charge regulator interposed between the electrical contacts and the battery, and may be configured in any number of alternative arrangements in alternative embodiments.

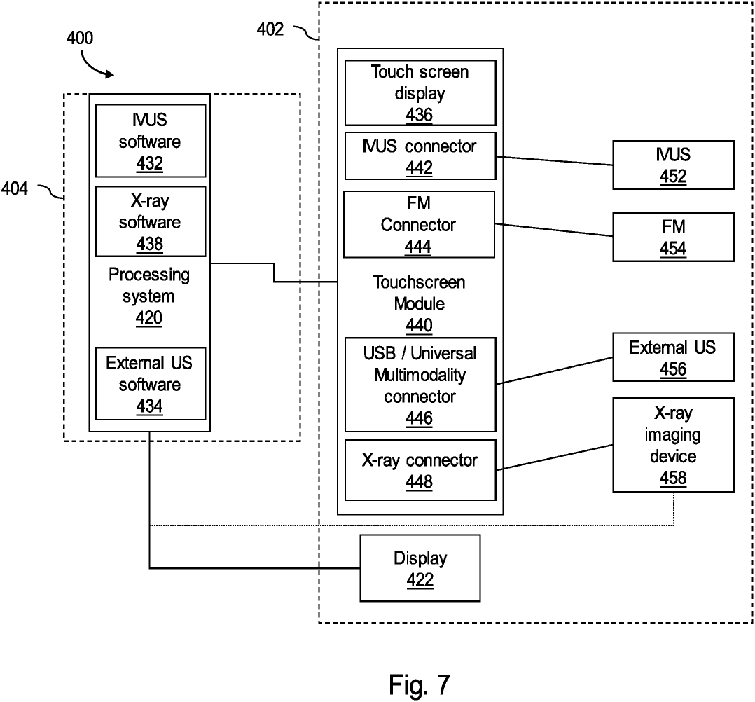

FIG. 7 is a diagrammatic schematic view of a multi-modality medical system 400 according to another embodiment of the present disclosure. Similar to the system 100 shown in FIG. 3, the system 400 shown in FIG. 7 is distributed between an imaging environment 402 and a control room 404. The system 400 is further configured to perform external X-ray imaging using an X-ray imaging device 458. The processing system 420 is in the control room 404, and is configured to control the various medical imaging and diagnostic modalities using software, including IVUS software 432, X-ray imaging software 438, and external ultrasound software 434. It will be understood that the processing system 420 may be configured to execute other software, such as the functional measurement FM device 454, which may include a pressure-sensing catheter or guidewire, or a flow-sensing catheter or guidewire.

The processing system 420 is communicatively coupled to a touchscreen module 440 in the imaging environment 402. The module 440 may provide a communication interface that includes a variety of connectors for connecting medical instruments of various different modalities to the processing system 420. In some embodiments, the module 440 may include some or all of the modality connectors included in the communication hub 101 illustrated in FIG. 4, for example. Further, the module 440 may be used as a bedside controller, similar to the controller 300 shown in FIGS. 6 and 7. In the illustrated embodiment, the module 440 further includes a touch screen display 436 configured to receive user inputs and display images and/or physiological data. The module 440 includes several connectors corresponding to several medical diagnostic modalities, including an IVUS connector 442, an FM connector 444, a USB /Universal multi-modality connector 446, and an X-ray connector 448. The X-ray imaging device 458 is coupled to the X-ray connector 448 of the module 440. Additionally, or alternatively, the X-ray imaging device 458 may be directly coupled to the processing system 420. The touch screen display 436 of the module 440 may be used to control an X-ray imaging procedure, such as an angiographic imaging procedure and/or a fluoroscopic imaging procedure. The X-ray imaging procedure may be performed simultaneously with a different diagnostic procedure, such as a pressure-sensing or IVUS pullback procedure to assess a stenosis within a blood vessel. The software 432, 438, 434 executed by the processing system 420 may be incorporated into a single software application. In other embodiments, the processing system 420 is configured to execute the different modalities using multiple software applications. In some embodiments, a different software application is used for each of the medical modalities.

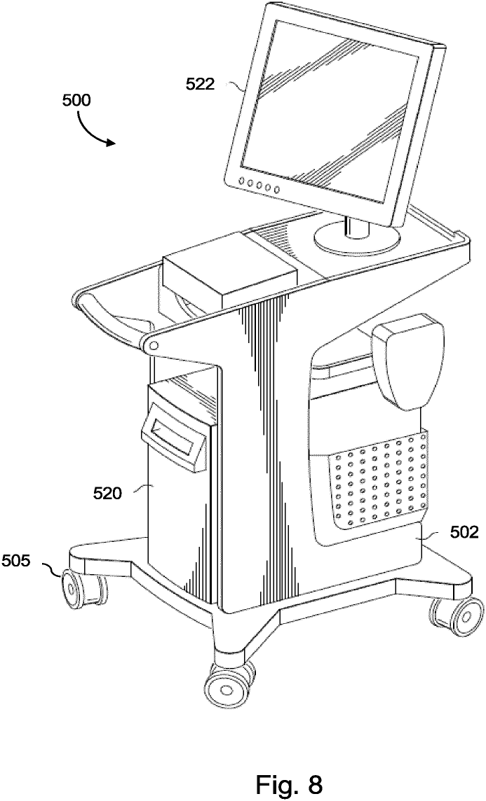

The systems 100, 400 described above may involve fixed systems that are integrated into a medical environment, such as a catheter lab 102, and a separate control room 104. The present disclosure also describes mobile, or portable multi-modality medical systems that provide the same advantages described above in a more portable platform. In this regard, FIG. 8 is a perspective view of a mobile multi-modality medical system 500. The system 500 includes a processing system 520, a display 522, and an interface device coupled to a mobile cart chassis 502. The mobile cart chassis 502 is movable through the medical environment by the wheels 505. As described further below, the system 500 may further include a communication interface configured to couple to a plurality of medical modalities, including imaging and physiological measurement modalities (e.g., pressure, flow, etc.) Accordingly, the system 500 may include some or all of the components described above with respect to the systems 100, 400. The interface device 536 is configured to receive user inputs to control one or more aspects of a medical diagnostic or therapeutic procedure. The user interface device 536 may include a touch screen interface, a keyboard, mouse, trackball, button, cursor, joystick, microphone, or any other suitable user interface device. The interface device 536 may be used, for example, to switch between the different medical modalities during a medical procedure.

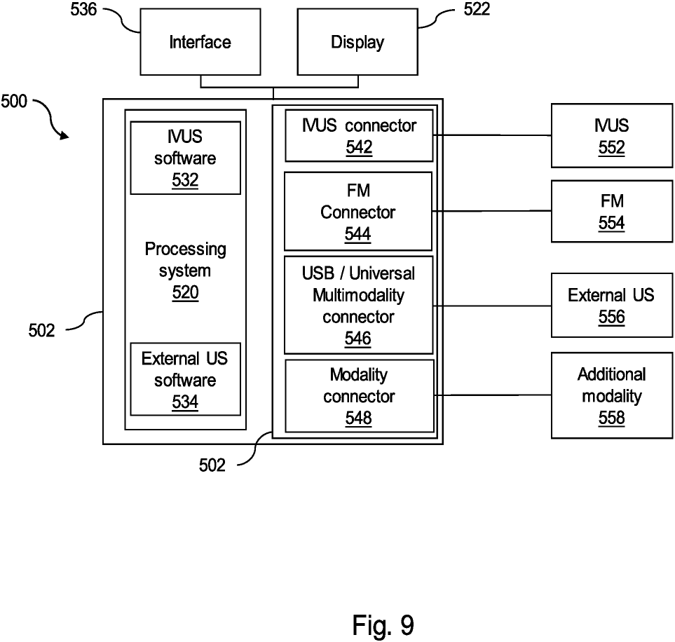

FIG. 9 is a diagrammatic schematic view of a portable multi-modality medical system 500. The system 500 includes a processing system 520 and communication interface 530. The processing system 520 and communication interface 530 are coupled to, or positioned within, a mobile chassis 502. The mobile chassis 502 may include a cart, such as the chart shown in FIG. 8. The communication interface 530 includes a plurality of connectors for different medical modalities, including an IVUS connector 542, and FM connector 544, a USB / Universal multi-modality connector 546, and an additional modality connector 548. The connectors are configured to be coupled to the corresponding medical modality, including an IVUS device 552, a functional measurement device 554, an external ultrasound imaging device 556, and a device of an additional modality 558. In some embodiments, the processing system 520 and the communication interface 530 are contained within a same housing. In other embodiments, the processing system 520 and the communication interface 530 are positioned within different housings. Similar to the systems 100, 400 described above, the system 500 incorporates external ultrasound 556 using a USB or universal multimodality connector 546. Using a universal-type interface standard for the ultrasound probe offers greater flexibility, as the USB connector 546 can be used for purposes other than ultrasound imaging when external ultrasound is not needed for a diagnostic or therapeutic procedure.

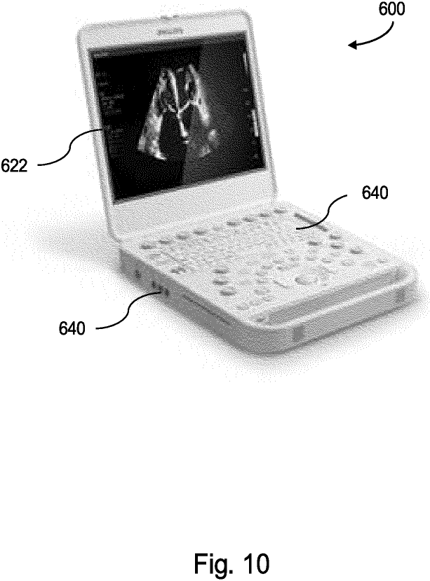

FIG. 10 is a perspective view of a mobile multi-modality medical system 600, according to a further embodiment of the present disclosure. In the illustrated embodiment, the system 600 comprises a clamshell-style computer design, similar to a laptop computer. The system 600 includes a plurality of interface devices 636, such as a keyboard, trackball, and/or a plurality of buttons, for controlling one or more medical modalities. The system 600 further comprises a plurality of connectors 640 corresponding to different medical modalities, which may include some or all of the connectors described above with respect to the systems 100, 400, and 500. For example, the system 600 may include connectors for IVUS imaging catheters and/or IVUS PIM devices, OCT imaging devices and/or PIM devices, pressure-sensing catheters and/or guidewires, intravascular flow-sensing instruments, ECG, external ultrasound, ICE imaging device, TEE probes, TTE probes, VH devices, and/or any other suitable medical modality. The system 600 further includes a display 622. The display 622 may comprise an LCD display, a touch screen display, or any other suitable type of display.

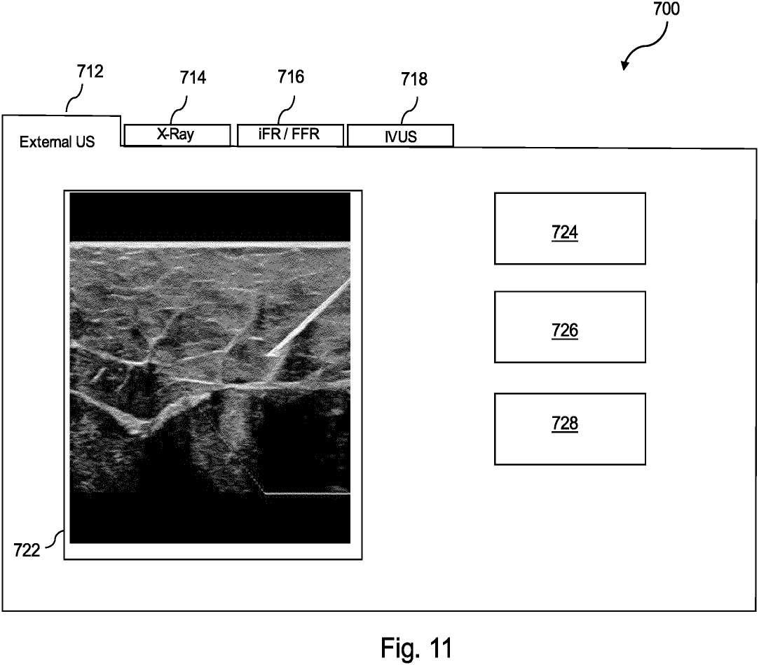

The systems described above may be configured to execute one or more software applications for guiding the different medical modalities. In some embodiments, a single application is used, and the operator can switch between different modalities using a graphical user interface provided by the application. In other embodiments, different software applications are used for one or more of the medical modalities. FIG. 11 illustrates a graphical user interface (GUI) 700 according to an embodiment of the present disclosure. The GUI 700 includes a plurality of toggles or tabs 712, 714, 716, 718, each tab associated with a different medical modality. By selecting a tab associated with a particular modality, the controls and/or data supplied by the modality can be observed on a window of the GUI 700. For example, in the illustrated embodiment, the tab 712 corresponding to the external ultrasound modality has been selected. The GUI 700 provides a view of the ultrasound data and a plurality of control interface objects 724, 726, 728. The ultrasound data is provided in a window 722. In some embodiments, the controls and data for multiple modalities are shown for one or more screens or window of the GUI 700. In some embodiments, selecting a different one of the tabs 712, 714, 716, 718, deactivates the previously selected modality, and initiates the controls and/or data collection for a different modality. In some embodiments, other toggles or interface objects can be used instead of or in addition to tabs for switching between the modalities, including buttons, drop-down list, a slider, radio buttons, or any other suitable user interface object.

In some embodiments, each of the tabs or windows is associated with a different application that can operate independently of the applications of the other modalities. Each application or module of the application may be available for selection on the screen display to provide for convenient switching between the modalities. In particular, the system may provide for convenient switching between an x-ray imaging modality, an intravascular imaging modality, a functional measurement modality, and/or an external ultrasound modality. The external ultrasound modality includes a self-sufficient ultrasound imaging device coupled to a communication interface, in some embodiments. The software application or applications may be executed by a processor of a processing system. In an exemplary embodiment, the processing system is configured to execute an external ultrasound imaging modality for inserting an intravascular device into the patient’s vasculature, and switch to a different modality based on a user input. For example, the processing system may be configured to control an external ultrasound probe to generate digital ultrasound data, cause a display to display ultrasound images based on the digital ultrasound data, and receive a user input signal from a user interface device representing a selection of an intravascular data acquisition mode. In response to receiving the user input the processing system is configured to control the intravascular device to generate intravascular data, and cause the display to display a graphical representation of the intravascular data.

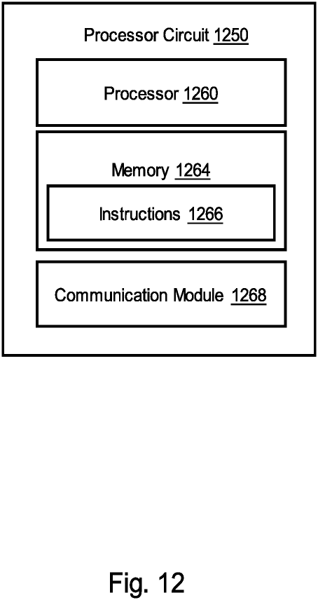

FIG. 12 is a schematic diagram of a processor circuit 1250, according to embodiments of the present disclosure. The processor circuit 1250 may be implemented in a Patient interface module, a processing system (e.g., 120, 420, 520), an ultrasound imaging device (156, 456, 556), a touch screen module, a communication interface or hub, or any other device described herein. As shown, the processor circuit 1250 may include a processor 1260, a memory 1264, and a communication module 1268. These elements may be in direct or indirect communication with each other, for example via one or more buses.

The processor 1260 may include a central processing unit (CPU), a digital signal processor (DSP), an ASIC, a controller, an FPGA, another hardware device, a firmware device, or any combination thereof configured to perform the operations described herein. The processor 1260 may also be implemented as a combination of computing devices, e.g., a combination of a DSP and a microprocessor, a plurality of microprocessors, one or more microprocessors in conjunction with a DSP core, or any other such configuration.

The memory 1264 may include a cache memory (e.g., a cache memory of the processor 1260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1264 includes a non-transitory computer-readable medium. The memory 1264 may store instructions 1266. The instructions 1266 may include instructions that, when executed by the processor 1260, cause the processor 1260 to perform the operations described herein with reference to the systems 100, 400, 500, 600 described herein. Instructions 1266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1268 can include any electronic circuitry and/or logic circuitry to facilitate direct or indirect communication of data between the devices of the different modalities described herein, a processing system, a communication interface, and/or a user interface device. In that regard, the communication module 1268 can be an input/output (I/O) device. In some instances, the communication module 1268 facilitates direct or indirect communication between various elements of the system described herein.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A multi-modality medical system, comprising:
an intravascular catheter or guidewire configured to output intravascular data;
an external ultrasound probe configured to output digital ultrasound data; and
a processor disposed within a multi-modality housing and configured for communication with the external ultrasound probe and the intravascular catheter or guidewire, wherein the processor is configured to:
in response to receiving a user input selecting an external ultrasound data acquisition mode:
control the external ultrasound probe to generate the digital ultrasound data; and
output, to a display in communication with the processor, an external ultrasound image based on the digital ultrasound data; and
in response to receiving a user input selecting an intravascular data acquisition mode:
control the intravascular catheter or guidewire to generate the intravascular data; and
output, to the display, a graphical representation based on the intravascular data.

2. The system of claim 1, further comprising:
a communication interface communicatively coupled to the processor, wherein the communication interface comprises a housing and at least one data bus connector, wherein the communication interface is configured to be coupled to the external ultrasound probe and an intravascular catheter or guidewire configured to obtain intravascular data; and
the display.

3. The system of claim 2, wherein the communication interface is configured to be spaced from the processor.

4. The system of claim 2, wherein the external ultrasound probe is coupled to the communication interface by an industry standardized data bus.

5. The system of claim 4, wherein the industry standardized data bus is a universal serial bus (USB) connection.

6. The system of claim 1, wherein the external ultrasound probe comprises an ultrasound transducer array and signal processing circuitry disposed within a housing, wherein the signal processing circuit is configured to receive analog signals from the ultrasound transducer array, and generate the digital ultrasound data based on the received analog signals, wherein the digital ultrasound data is configured to be processed into images and displayed using non-ultrasound-specific hardware.

7. The system of claim 6, wherein the signal processing circuitry of the external ultrasound probe comprises:
an analog-to-digital converter; and
a beamformer.

8. The system of claim 6, wherein the signal processing circuitry is configured to process raw ultrasound signals to generate the digital ultrasound data such that the digital ultrasound data is in an industry standard image format or video format.

9. The system of claim 6, wherein the signal processing circuitry is configured to perform at least one of envelope detection, log compression, or scan conversion.

10. The system of claim 6, wherein the external ultrasound probe further comprises a data bus connector coupled to the external ultrasound probe by a cable, wherein the cable is permanently connected to the housing of the external ultrasound probe.

11. The system of claim 6, wherein the external ultrasound probe further comprises:
a data bus connector coupled to the housing of the external ultrasound probe; and
a cable comprising a matching data bus connector configured to removably couple to the data bus connector coupled to the housing.

12. The system of claim 1, further comprising a user interface device, wherein the user interface device comprises a further display, wherein the processor is configured to cause the further display to display a graphical user interface that includes a first toggle associated with the external ultrasound data acquisition mode, and a second toggle associated with the intravascular data acquisition mode, and wherein the user interface device is configured to generate a user input signal based on a selection by a user of the first toggle or the second toggle.

13. The system of claim 12, further comprising an X-ray imaging device, wherein the processor is configured to:
receive a further user input selecting an X-ray imaging mode; and
in response to receiving the further user input:
control the X-ray imaging device to generate X-ray image data; and
output, to the display, an X-ray image based on the X-ray image data.

14. The system of claim 13, wherein the processor is further configured to co-register the intravascular data with the X-ray image data.

15. The system of claim 12, wherein the further display comprises a touch screen display configured to receive the selection from the user.

16. The system of claim 12, wherein the processor is configured to cause the display and the further display to simultaneously display the graphical representation based on the intravascular data.

17. The system of claim 1, wherein the intravascular catheter or guidewire comprises an intravascular ultrasound (IVUS) imaging catheter, and wherein the intravascular data comprises IVUS images.

18. The system of claim 1, wherein the intravascular catheter or guidewire comprises at least one of an intravascular pressure sensor or an intravascular flow sensor, and wherein the intravascular data comprises at least one of pressure data or flow data.

19. A multi-modality medical system, comprising:
a hand-held external ultrasound probe comprising:
a first housing configured to be grasped by a human hand;
an ultrasound transducer array coupled to the first housing; and
signal processing circuitry disposed within the first housing, wherein the signal processing circuit is configured to receive analog signals from the ultrasound transducer array, and generate digital ultrasound data based on the received analog signals, wherein the digital ultrasound data is configured to be processed into images and displayed using non-ultrasound-specific hardware; and
a first standard bus connector;
a touch screen interface device;
a display;
a communication hub comprising:
a second housing;
a second standard bus connector coupled to the second housing, wherein the second standard bus connector is configured to be coupled to the first standard bus connector of the external ultrasound probe; and
an intravascular modality bus connector coupled to the second housing, wherein the intravascular modality bus connector is configured to be coupled to an intravascular device; and
a processing system comprising a processor and a memory, wherein the processing system is configured to be coupled to the communication hub, the touch screen interface device, and the display, wherein the processing system is in communication with the external ultrasound probe and the intravascular device via the communication hub, and wherein the memory comprises instructions executable by the processor to:
control the external ultrasound probe to generate the digital ultrasound data;
cause at least one of the touch screen interface device or the display to display ultrasound images based on the digital ultrasound data;
receive a user input signal from the touch screen interface device, wherein the user input signal represents a selection of an intravascular data acquisition mode; and
in response to receiving the user input signal:
control the intravascular device to generate intravascular data; and
cause at least one of the touch screen interface device or the display to display a graphical representation of the intravascular data.
